(19) United States
(12) Patent Application Publication
(10) Pub. No.: US 2021/0022631 A1
(43) Pub. Date: Jan. 28, 2021

(54) AUTOMATED OPTIC NERVE SHEATH DIAMETER MEASUREMENT

(71) Applicant: The Regents of the University of Michigan, Ann Arbor, MI (US)

(72) Inventors: Sayedmohammadreza Soroushmehr, Ann Arbor, MI (US); Kayvan Najarian, Northville, MI (US); Venkatakrishna Rajajee, Ann Arbor, MI (US); Kevin Ward, Ann Arbor, MI (US); Jonathan Gryak, Ann Arbor, MI (US); Craig A. Williamson, Ann Arbor, MI (US); Mohamad H. Tiba, Ann Arbor, MI (US)

(21) Appl. No.: 16/554,138

(22) Filed: Aug. 28, 2019

Related U.S. Application Data
(60) Provisional application No. 62/877,539, filed on Jul. 23, 2019.

(51) Int. Cl. A61B 5/03; A61B 5/00; A61B 8/08

(52) U.S. Cl. CPC A61B 5/031; A61B 5/7225; G06K 9/6223; A61B 8/0808; A61B 8/5223

(57) ABSTRACT

A method of determining a diameter of a sheath of an optic nerve includes obtaining, by a processor, scan data representative of the optic nerve sheath, analyzing, by the processor, the scan data to find a position of a globe-optic nerve interface point, segmenting, by the processor, the scan data, processing, by the processor, the segmented scan data at an offset from the position of the globe-optic nerve interface point to determine boundary positions of the optic nerve sheath, and calculating, by the processor, the diameter of the optic nerve sheath based on the determined boundary positions.

### CROSS-REFERENCE TO RELATED APPLICATION

This application claims the benefit of U.S. provisional application entitled “Automated Optic Nerve Sheath Diameter Measurement,” filed Jul. 23, 2019, and assigned Serial No. 62/877,539, the entire disclosure of which is hereby expressly incorporated by reference.

### STATEMENT REGARDING FEDERALLY SPONSORED RESEARCH OR DEVELOPMENT

This invention was made with government support under Contract No. W81XWH-18-1-0005 awarded by the United States Army Medical Research and Materiel Command (USAMRMC). The government has certain rights in the invention.

### BACKGROUND OF THE DISCLOSURE

### Field of the Disclosure

The disclosure relates generally to assessment of intracranial pressure based on automated measurement of optic nerve sheath diameter.

### Brief Description of Related Technology

The optic nerve is a part of the central nervous system. The optic nerve is surrounded by cerebrospinal fluid and is encased in a sheath. The optic nerve sheath is an anatomical extension of the duramater, the outermost and most substantial meningeal layer of the central nervous system. The subarachnoid space around the optic nerve is continuous with the intracranial subarachnoid space, and is surrounded by cerebrospinal fluid (CSF).

Changes in CSF pressure can result from brain injury, tumor rupture, and other conditions. Changes in CSF pressure can, in turn, reflect changes in intracranial pressure (ICP). The degree of elevation and duration of elevated ICP are correlated with patient outcomes. Therefore, ICP monitoring can provide useful information for patients' management and treatment, and is widely used in the management of patients with severe traumatic brain injury (TBI).

However, ICP monitoring is an invasive monitoring procedure. ICP monitoring can thus cause complications, such as intracranial hemorrhage, dislocation, and infection. Moreover, direct measurement of ICP, especially for patients with minor brain injury, is an unrealistic and aggressive requirement.

Some clinical trials and research studies have attempted to replace the ICP monitoring with a non-invasive alternative measurement. It has been shown that the diameter of the optic nerve sheath changes rapidly with changes in CSF pressure. For instance, studies have shown that ventriculostomy measurements of intracranial pressure are correlated with ultrasound (US) optic nerve sheath diameter measurements. The optic nerve sheath diameter may thus be used as a non-invasive test for elevated ICP. Unfortunately, manual techniques for measuring optic nerve sheath diameter are often and typically tedious, time-consuming, and subject to human error.

### SUMMARY OF THE DISCLOSURE

In accordance with one aspect of the disclosure, a method of determining a diameter of a sheath of an optic nerve includes obtaining, by a processor, scan data representative of the optic nerve sheath, analyzing, by the processor, the scan data to find a position of a globe-optic nerve interface point, segmenting, by the processor, the scan data, processing, by the processor, the segmented scan data at an offset from the position of the globe-optic nerve interface point to determine boundary positions of the optic nerve sheath, and calculating, by the processor, the diameter of the optic nerve sheath based on the determined boundary positions.

In accordance with another aspect of the disclosure, a system of determining a diameter of an optic nerve sheath includes a memory in which scan data input instructions, scan data analysis instructions, segmentation instructions, and boundary identification instructions are stored, and a processor in communication with the memory and configured to, upon execution of the scan data input instructions, obtain scan data representative of a two-dimensional slice through the optic nerve sheath and a globe from which the optic nerve extends, upon execution of the scan data analysis instructions, analyze the scan data to find an anterior-posterior position of a globe-optic nerve interface point, upon execution of the segmentation instructions, implement a segmentation procedure to generate a super-pixel representation of the scan data, and upon execution of the boundary identification instructions, process the super-pixel representation of the scan data at an offset from the anterior-posterior position of the globe-optic nerve interface point to determine boundary positions of the optic nerve sheath, and calculate the diameter of the optic nerve sheath based on the determined boundary positions.

In accordance with yet another aspect of the disclosure, a computer readable storage medium having stored therein data representing instructions executable by a programmed processor for determining a diameter of an optic nerve sheath, the storage medium including instructions for obtaining scan data representative of a two-dimensional slice through the optic nerve sheath and a globe from which the optic nerve extends, analyzing the scan data to find a position of a globe-optic nerve interface point, implementing a segmentation procedure, the segmentation procedure being configured to generate a super-pixel representation of the scan data, processing the super-pixel representation of the scan data at an offset from the anterior-posterior position of the globe-optic nerve interface point to determine boundary positions of the optic nerve sheath, and calculating the diameter of the optic nerve sheath based on the determined boundary positions.

In connection with any one of the aforementioned aspects, the systems, storage media, and/or methods described herein may alternatively or additionally include or involve any combination of one or more of the following aspects or features. Processing the segmented scan data includes finding peaks in the segmented scan data at the offset, and determining a location of a minimum between the found peaks. Processing the segmented scan data includes processing the segmented scan data includes computing a derivative of the segmented scan data at the offset. Processing the segmented scan data includes determining a lateral position of the globe-optic nerve interface point at the offset based on a minimum between peaks in the segmented scan data, computing a derivative of the segmented scan data at the offset, and finding a pair of peaks in the derivative of the segmented scan data, each peak of the pair of peaks being disposed on a respective side of the lateral position. Finding the first and second peaks includes disregarding peaks in the derivative greater than a threshold. Finding the first and second peaks further includes, after disregarding the peaks greater than the threshold, finding a negative peak closest to the lateral position of the globe-optic nerve interface point, and finding a positive peak closest to the lateral position of the globe-optic nerve interface point. Analyzing the scan data includes computing a line integral of the scan data at each anterior-posterior position of the scan data, and finding a maximum of the line integral to determine the position of the globe-optic nerve interface point. The line integral is a first line integral, the method further including computing a second line integral of the scan data at each lateral position of the scan data, and determining a subset of the scan data corresponding with a region of interest based on the first line integral and the second line integral. Segmenting the scan data is implemented on the determined subset of the scan data. The scan data includes a plurality of frames. The method further includes disregarding one or more frames of the plurality of frames based on whether the first and second line integrals present peaks indicative of the globe and the optic nerve such that analyzing the scan data, segmenting the scan data, processing the segmented scan data, and calculating the diameter are repeated for the scan data of each remaining frame of the plurality of frames. The scan data includes two-dimensional slice data, the two-dimensional slice data being representative of a slice through the globe and the optic nerve. Processing the segmented scan data includes selecting a line of the scan data located about 3 millimeters in a posterior direction from the globe-optic nerve interface point as a subset of the segmented scan data at the offset to be processed. Obtaining the scan data includes capturing ultrasound scan data, cropping the ultrasound data, and removing noise from the cropped ultrasound scan data to generate the scan data. Removing the noise includes implementing a filtering procedure configured to preserve edges in the cropped ultrasound scan data. The scan data includes a plurality of frames. Analyzing the scan data, segmenting the scan data, processing the segmented scan data, and calculating the diameter are repeated for the scan data of each frame of the plurality of frames, and the method further includes compiling the calculated diameters of the optic nerve sheath for the plurality of frames to determine a value for the diameter of the optic nerve sheath. A method of determining an assessment of intracranial pressure including the method as described herein, and further including determining an intracranial pressure level based on the value for the diameter and based on a database correlating diameter values with corresponding levels of intracranial pressure. The segmentation procedure includes a k-means clustering procedure. The execution of the segmentation instructions further configures the processor to discard super-pixels below a threshold size. The execution of the boundary identification instructions further configures the processor to determine a lateral position of the globe-optic nerve interface point at the offset based on a minimum between peaks in the super-pixel representation of the scan data, compute a derivative of the super-pixel representation of the scan data at the offset, and find a pair of peaks in the derivative of the super-pixel representation of the scan data, each peak of the pair of peaks being disposed on a respective side of the lateral position. Processing the super-pixel representation of the scan data includes determining a lateral position of the globe-optic nerve interface point at the offset based on a minimum between peaks in the super-pixel representation of the scan data, computing a derivative of the super-pixel representation of the scan data at the offset, and finding a pair of peaks in the derivative of the super-pixel representation of the scan data, each peak of the pair of peaks being disposed on a respective side of the lateral position.

### BRIEF DESCRIPTION OF THE DRAWING FIGURES

For a more complete understanding of the disclosure, reference should be made to the following detailed description and accompanying drawing figures, in which like reference numerals identify like elements in the figures.

FIG. 1 is a flow diagram of a method of determining optic nerve sheath diameter in accordance with one example.

FIG. 2 is a rendered image of ultrasound scan data of a two-dimensional, sagittal slice of a globe and optic nerve that may be used by the method of FIG. 1 to determine the optic nerve sheath diameter in accordance with one example.

FIG. 3 depicts plots of two line integrals superimposed on ultrasound scan data from which the line integrals are computed in the method of FIG. 1 in accordance with one example.

FIG. 4 is a rendered image of ultrasound scan data (e.g., raw ultrasound scan data) before implementation of a filtering procedure of the method of FIG. 1 in accordance with one example.

FIG. 5 is a rendered image of the ultrasound scan data of FIG. 4 after implementation of a filtering procedure of the method of FIG. 1 in accordance with one example.

FIG. 6 is a rendered image of super-pixels generated from the filtered ultrasound scan data of FIG. 5 via implementation of a segmentation procedure of the method of FIG. 1 in accordance with one example.

FIG. 7 depicts plots of (1) intensity levels of a line (e.g., row) of super-pixels generated via implementation of a segmentation procedure of the method of FIG. 1 in accordance with one example, and (2) values of the derivative of the intensity levels as computed in the method of FIG. 1 in accordance with one example.

FIG. 8 is a block diagram of a system of determining optic nerve sheath diameter in accordance with one example.

The embodiments of the disclosed systems and methods may assume various forms. Specific embodiments are illustrated in the drawing and hereafter described with the understanding that the disclosure is intended to be illustrative. The disclosure is not intended to limit the invention to the specific embodiments described and illustrated herein.

### DETAILED DESCRIPTION OF THE DISCLOSURE

Methods and systems of automated measurement of optic nerve sheath diameter are described. Methods and systems for assessing intracranial pressure based on the calculated diameter are also described. The disclosed methods and systems measure the sheath diameter via analysis of scan data, such as ultrasound scan data. The analysis may include image segmentation and other image processing. In some cases, the image segmentation procedure includes or involves super-pixel analysis. The automated nature of the disclosed methods and systems avoids the errors of the manual measurement techniques. The disclosed methods and systems may also be useful in supporting further study of ICP and other monitoring.

The image segmentation and/or other aspects of the disclosed methods and systems address a number of challenges arising from the use of scan data, such as ultrasound scan data, to measure the sheath diameter. For instance, the ultrasound scan data may have a low signal to noise ratio (SNR), low contrast, and/or blurry boundaries. In some cases, filtering and/or other preprocessing steps are used to remove noise while maintaining edges and other boundaries. Additionally or alternatively, restricting the image segmentation to a region of interest and/or otherwise cropping the scan data may be used to address the challenges arising from the nature of the ultrasound scan data.

A number of different segmentation procedures may be used to segment the ultrasound scan data. Suitable segmentation procedures include those used on ultrasound scan data for applications involving left ventricle analysis, cancer screening, obstetrics and gynecology, and vascular disease. The segmentation procedures may use a wide variety of information, including, for instance, various image features (e.g., gray level distribution, intensity gradient, phase and texture), shape information, and temporal information (e.g., for those containing image sequences). The image segmentation procedures implemented by the disclosed methods and systems may avoid relying on complex processing techniques (e.g., convolutional neural networks), due to, for instance, the recognition that the consecutive images in the ultrasound video sequence are correlated. Notwithstanding the correlated nature of the images, those and other segmentation techniques may nonetheless be used by the disclosed methods and systems in some cases.

The disclosed methods and systems may use and process temporal information to determine the sheath diameter from image frames throughout an ultrasound video sequence. The diameters calculated for each image frame may then be compiled to arrive at a final value via voting and/or other statistical computation(s).

Although described in connection with scan data captured as a two-dimensional ultrasound video, the disclosed methods and systems may be applied to a wide variety of scan data. Other types of ultrasound scan data may be used, including, for instance, three-dimensional ultrasound scan data. Other types of imaging modalities may also be used to capture the scan data, including, for instance, magnetic resonance imaging. The formatting and other characteristics of the scan data may also vary. The characteristics of the scan data may also result in a modification of one or more aspects of the disclosed methods and systems, including, for instance, image de-noising or other procedure step in the image processing, or an act in the disclosed method.

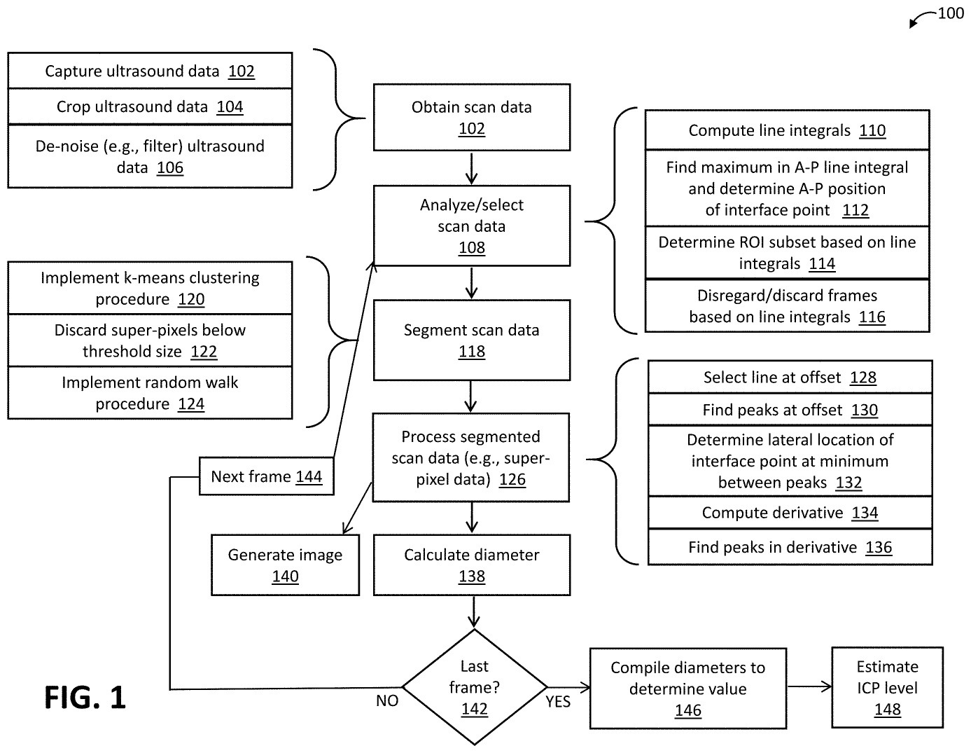

FIG. 1 depicts a method 100 of determining (e.g., measuring) a diameter of a sheath of an optic nerve. The method 100 may be implemented by a processor, such as an image processor. The processor may or may not be part of an imaging system, such as an ultrasound imaging system. In some cases, the method 100 is implemented by a processor configured via execution of instructions stored on a computer-readable storage medium.

The method 100 includes an act 102 in which scan data is obtained. The scan data is representative of the optic nerve, including the optic nerve sheath, and a globe of the eye from which the optic nerve extends. In some cases, the scan data is or includes two-dimensional slice data. The slice data is representative of a slice through the optic nerve and the globe. The slice may be oriented as a sagittal slice. Alternative or additional types of scan data may be obtained. For instance, the scan data may be or include three-dimensional scan data.

The scan data may be or include ultrasound scan data. In such cases, obtaining the scan data may include capturing ultrasound scan data in an act 102. The raw data generated by an ultrasound imaging system may then be processed (e.g., pre-processed). For example, the pre-processing may include cropping the raw ultrasound data in an act 104 and/or removing noise from the ultrasound data in an act 106. An example of raw ultrasound data representative of the globe and optic nerve is shown in FIG. 4.

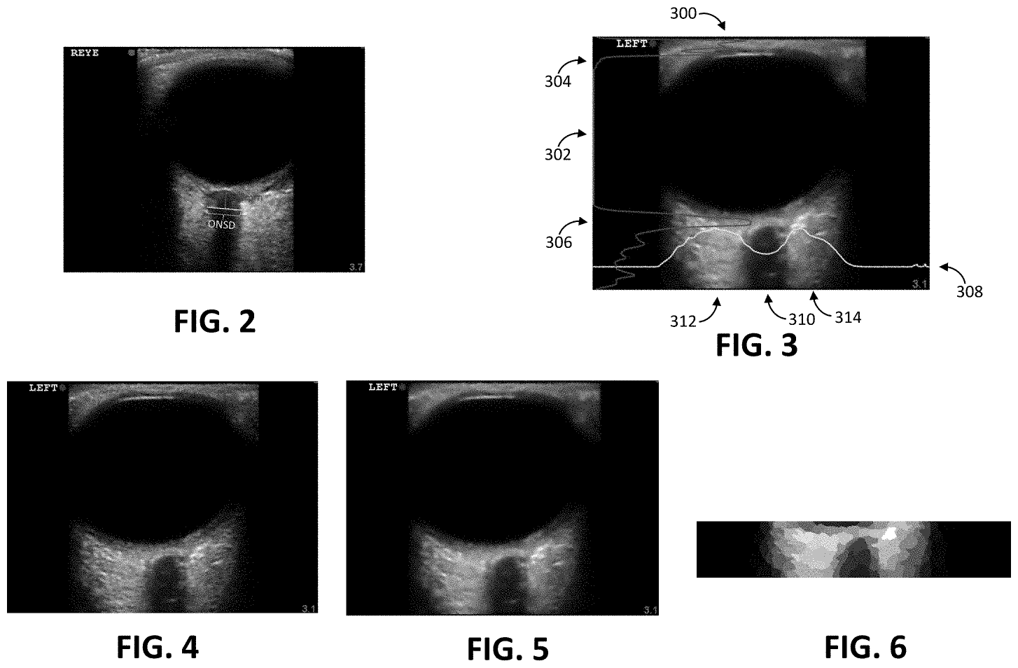

Cropping may be implemented via Digital Imaging and Communications in Medicine (DICOM) attributes (e.g., metadata) that specify, for instance, the location of the entire scan containing the nerve sheath and the retinal detachment. In some cases, the cropping may be result in scan data limited to depicting the globe and a portion of the optic nerve. An example of a cropped frame of scan data is shown in FIG. 2, in which the diameter of the optic nerve sheath is labeled ONSD at a position offset from the interface with the globe. A variety of other cropping techniques may be used. DICOM metadata may alternatively or additionally be relied upon to provide information regarding the resolution of image, such as how many pixels correspond with a millimeter (mm).

De-noising and/or other resolution enhancement of the ultrasound data in the act 106 of FIG. 1 may be achieved via implementation of one or more filtering procedures. The filtering may be configured to preserve edges in the cropped ultrasound scan data, such as the edges of the globe and the optic nerve sheath. In some cases, image guided filtering is used to filter the scan data while preserving edges. In image guided filtering, the filtering input image and guidance image are shown as p and I respectively. The images are divided to overlapped windows with radius of r and following coefficients are computed in each window:

$${a}_{k} = \frac{c o {v}_{k} ( I , P )}{v a {r}_{k} ( I ) + ɛ} \\ {b}_{k} = {\overline{p}}_{k} - {a}_{k} {\overline{I}}_{k}$$

where k is the window index, Iₖ and pₖ are average of intensities in k^{th} window in noisy and guidance images, respectively. Also ε is a regularization parameter that determines the edge-preserving property of the filter. The filtered pixel qᵢ is the average of aₖIᵢ+bₖ in all the windows that cover qᵢ. The coy and var functions compute the covariance and variance, respectively. FIG. 5 shows the raw ultrasound data of FIG. 4 after de-noising.

Alternative or additional types of filtering procedures may be implemented to de-noise the scan data while preserving edges. For example, a Savitzky-Golay filter as described in Chinrungrueng et al. “Fast edge-preserving noise reduction for ultrasound images” IEEE Transactions on Nuclear Science, 48(3), pp. 849-854 (2001), may be used.

Fewer, additional, or alternative pre-processing steps may be implemented in connection with obtaining the scan data. For instance, cropping the ultrasound data at this stage of the method 100 may not be necessary in some cases. For example, cropping may be effectively implemented later in the method 100 in connection with selection of a subset of the scan data, as described further below.

In an act 108, the scan data is analyzed to find a position of a globe-optic nerve interface point. The act 108 may be directed to finding the position of the interface point in the anterior-posterior dimension or direction. The analysis may be directed to selecting subsets of the scan data. The subsets may correspond with a subset of the frames and/or with a cropped portion of each frame.

In the example of FIG. 1, the analysis of the scan data includes computing line integrals of the scan data in an act 110. An anterior-posterior (AP) line integral may involve summing the scan data at each anterior-posterior position of the scan data. An example of the AP line integral is depicted in FIG. 2 as signal 300. In that example, the AP line integral is a vertical signal v that has a minimum 302 within the globe and maxima 304, 306 at either edge of the globe. In an act 112, the maximum 306 of the AP line integral may be used to determine the AP position, or vertical pixel, of the point at which the globe and the optic nerve meet, i.e., the globe-optic nerve interface point.

Another line integral may also be computed in the act 110 at each lateral position of the scan data. The lateral line integral is depicted in FIG. 2 as signal 308, a horizontal signal h having a minimum 310 at the optic nerve and two peaks 312, 314 that establish a region of interest encompassing the optic sheath. The lateral line integral may be used to determine the lateral position, or horizontal pixel, of the globe-optic nerve interface point.

The integrals may be calculated via a summation of pixel values of the image array in each column and each row separately. If the denoised image is an N×M image shown as I_{d}, then the line integrals are computed as the following one-dimensional signals.

v(n)=Σₘ₌₁^{M}I_{d}(n, m) for n=1, . . . , N

h(m)=Σₙ₌₁^{N}I_{d}(n, m) for m=1, . . . , M

An example of the v signal is depicted as the signal 308 of FIG. 3. The v signal is a result of the vertical line, or column, integrals, and has two main peaks 312, 314, corresponding to the brighter regions and a local minimum 310 between the peaks 312, 314 corresponding to the dark region inside the sheaths. The peaks 312, 314 may be used to identify or define the region of interest, as described below. For instance, if the minimum of the v signal is the g^{th} element of the signal, then the value of the v signal corresponds to the column at which the globe is located.

$$g = {argmin}_{M {ax}_{1} \leq i \leq M {ax}_{2}} v ( i )$$

An example of the h signal is depicted as the signal 300 of FIG. 3. The h signal is a result of the horizontal line, or row, integrals, and is used to identify the globe-optic nerve interface point.

In an act 114, the AP and lateral line integrals may also be used to determine a subset of the scan data corresponding with a region of interest. The region of interest may only include a portion of the optic nerve, but the region of interest may vary. The scan data may thus be further cropped based on the line integrals to a region of interest. The region of interest may, for example, correspond with only relevant portions of the globe and optic nerve. Further image processing, such as image segmentation, may then be implemented on the subset of the scan data.

One or both of the line integrals may be used to focus, filter, or reduce the scan data down to a subset in alternative or additional ways. For example, the line integrals may be used to select which images should be further processed and relied upon to measure the optic nerve sheath diameter. In some ultrasound examples, the scan data includes a plurality of frames of the ultrasound video. In such cases, the analysis may include an act 116 in which one or more frames are discarded or otherwise disregarded based on whether the line integrals indicate that the frame has suitably captured the globe and optic nerve. For example, the act 116 may include analyzing each frame to determine whether the line integrals present peaks indicative of the globe and the optic nerve. For each such qualifying frame, the remaining acts of the method 100 may then be repeated as described below.

The method 100 includes an act 118 in which the scan data is segmented. In the example of FIG. 1, the segmentation may occur after finding the interface point and defining the region of interest (ROI) subset of the scan data. The segmentation may generate super-pixels of the scan data. For example, a super-pixel segmentation procedure, such as simple linear iterative clustering (SLIC) may be used in an act 120 to segment the scan data to super-pixels. The SLIC segmentation procedure includes a k-means clustering procedure implemented in which the image is partitioned into homogenous regions based on the k-means clustering technique. In that technique, the scan data of the image is first partitioned to non-overlapped blocks/tiles, and the center of each tile is used as an initial parameter for clustering. After that, the center of each tile is refined and also its shape is modified in an iterative process using the Lloyd algorithm. The modified shape is the super-pixel.

As part of the SLIC procedure or otherwise, the super-pixels may then be analyzed in an act 122 in terms of area, in which super-pixels below a threshold size are excluded from the results or otherwise discarded. Alternative or additional image segmentation procedures may be implemented. For example, a random walks segmentation procedure may be implemented in an act 124.

FIG. 6 depicts the ultrasound data of FIGS. 4 and 5 in the region of interest after implementation of SLIC segmentation.

Returning to FIG. 1, in an act 126, the segmented scan data (e.g., the super-pixel data) is processed to determine positions of boundaries of the optic nerve sheath. The processing is implemented at an offset from the position of the globe-optic nerve interface point. The offset is in the posterior direction, away from the interface point. In some cases, the offset is about 3 mm, but other offset amounts may be used.

The processing of the segmented scan data may include an act 128 in which a line of the scan data is selected. The selection may include determining how many pixels correspond with the 3 mm (or other) offset amount. The selection then determines a subset of the segmented scan data to be processed. For example, a single row of pixels may be selected. Alternatively, multiple rows of pixels are selected.

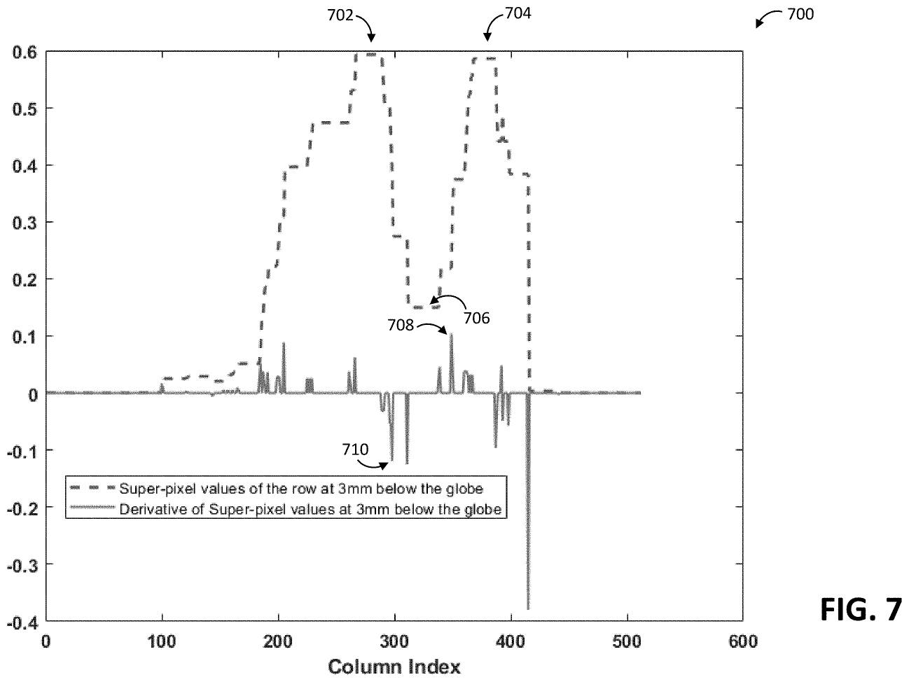

The processing of the segmented scan data may include finding, in an act 130, a number of peaks in the segmented scan data selected in the act 128. For example, the positions (e.g., lateral positions) of a pair of peaks in the row at the offset (e.g., the 3 mm row) may be located. The row at the offset may be determined based on the size of each pixel, which can be extracted from DICOM metadata. An example of the segmented scan data at the offset is depicted in a plot 700 of FIG. 7. The processing may include analysis of the peaks and derivative of this row of super-pixel data. A first intensity peak 700 is located at about column index 280 and a second intensity peak is located at about column index 370. Each column index may correspond with a pixel number in the lateral direction.

The lateral location of a minimum between the found peaks may then be found or otherwise determined in an act 132 (FIG. 1). The minimum may correspond with the lateral location or position of the globe-optic nerve interface point. In the example of FIG. 7, a minimum 706 between the pair of peaks 702, 704 is located at about column index 320. The lateral position of the globe-optic nerve interface point may be based on the minimum between peaks in the segmented scan data in alternative or additional ways. For example, the minimum location or position may be alternatively or additionally determined by finding a midpoint between the pair of peaks 702, 704.

Processing the segmented scan data may include an act 134 in which a derivative of the segmented scan data at the offset is computed. The derivative may be calculated by subtracting each intensity value from the previous one. FIG. 7 depicts a plot of an example of the computed derivative.

A pair of peaks in the derivative may then be found in an act 136. Each peak is disposed on a respective side of the lateral position of the minimum 706, i.e., the globe-optic nerve interface point.

Finding the peaks in the derivative may be subject to one or more rules, conditions, or other guidelines. For instance, a peak in the derivative may be disqualified if the magnitude of the derivative exceeds a predetermined threshold. Alternatively or additionally, peaks below a floor may be disregarded as noise. Finding the peaks may thus be configured to find the first significant peaks reached from the minimum. Other conditions or guidelines may be applied or considered. For example, the distance between the globe-optic nerve interface point and the peak should fall within a predetermined range.

Whether the derivative is positive or negative may also be used to select the peaks. For example, the first significant positive peak closest to, and after (i.e., to the right of), the globe-optic nerve interface point, and the first significant negative peak closest to, and before (i.e. to the left of), the globe-optic nerve interface point, may be selected. In the example of FIG. 7, first significant positive and negative peaks 708, 710 in the derivative curve are at about column indices 330 and 290, respectively.

The diameter of the optic nerve sheath is then calculated in an act 138 based on the determined positions of the boundaries. For example, in connection with the data depicted in FIG. 7, the diameter is calculated as the distance corresponding to the difference between the column indices 290 and 330.

In some cases, an image of the scan data and/or segmented scan data from which the diameter is measured is generated in an act 140. The act 140 may, for example, include rendering an image on a display. The image may be rendered or otherwise generated at other times during implementation of the method 100. For instance, the image may be rendered before the processing of the act 126 to provide an operator an opportunity to discard the scan data of a frame, thereby removing some of the scan data from the measurement.

The method 100 may include a decision block 142 to determine whether a last frame has been processed. For example, the last frame may be final frame in an ultrasound video or other sequence of images. If not, control passes to a block 144 in which the next frame of scan data is selected. In the example of FIG. 1, some or all of the pre-processing and analysis of the act 108 is then implemented. For example, the frames may be separately cropped, pre-qualified, and/or otherwise pre-processed in preparation for segmentation. Computation and/or analysis of the line integrals to determine whether the scan data for the frame is suitable may also be performed for the next frame. In other cases, control may return to alater step in the method 100, such as implementation of the segmentation procedure. Either way, in cases in which the scan data includes a plurality of frames, the above-described analysis, segmentation, and processing may be repeated to measure the sheath diameter for each frame.

Once the last frame has been processed, control passes to an act 146 in which the diameter measurements for all of the frames are compiled to determine a value, e.g., a final value for the ultrasound video. In some cases, the compilation involves a voting procedure. For example, a median value may be determined. Other voting procedures or other techniques for the determination may be used. For example, one or more statistical procedures may be used to filter the measurements before finding the median or otherwise implementing a voting procedure.

In the example of FIG. 1, the final measurement value for the sheath diameter is used in an act 148 to determine an assessment of a corresponding intracranial pressure (ICP) level. In some cases, the corresponding ICP level is estimated via a look-up table or other database correlating sheath diameters and ICP levels. The ICP level may be estimated from such correlation data via interpolation. Alternatively or additionally, the ICP level may be computed as a function of the sheath diameter, the function being or including a polynomial expression fit to the data.

The method 100 may include one or more additional acts. In one example, one or more acts are directed to providing the measurement value as an output. Alternatively or additionally, the method 100 may be repeated, e.g., daily, hourly or otherwise, to see if the sheath diameter is increasing or changing. Such repetition is not problematic because the method 100 is non-invasive, not painful, and otherwise not undesirable or troubling for the patient.

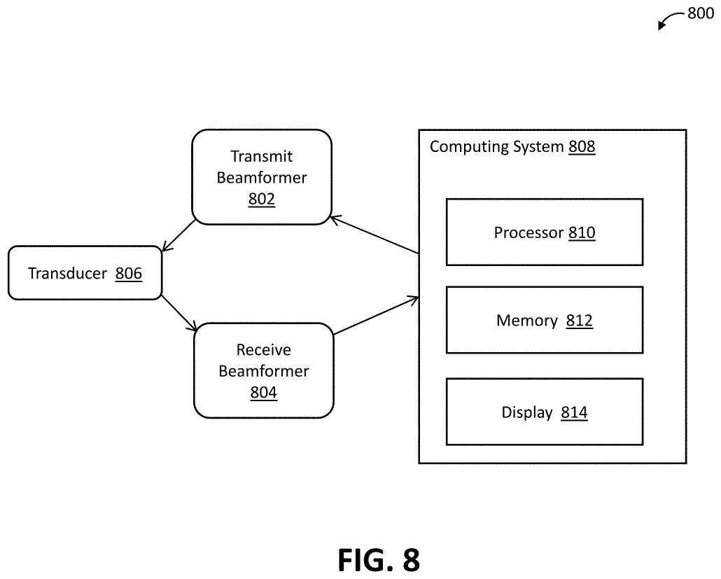

FIG. 8 depicts a system 800 of determining a diameter of an optic nerve sheath and/or ICP level based on the sheath diameter. The system 800 may be used to implement the methods described above, and/or a different method. The system 800 may also be used to determine the sheath diameter and/or ICP level via execution of one or more sets of instructions, as described below.

The system 800 may be or include an imaging system. In the example of FIG. 8, the system 800 includes an ultrasound imaging system having a transmit beamformer 802, a receive beamformer 804, and a transducer 806. Additional, fewer, or alternative imaging system components may be provided. For instance, the system 800 may not include the front-end components of the imaging system. Thus, in some cases, the system 800 is a medical diagnostic ultrasound system. In other cases, the system 800 is a computer or workstation.

The transducer 806 is an array of elements. For example, the elements are piezoelectric or capacitive membrane elements. The array is configured as a one-dimensional array, a two-dimensional array, a 1.5D array, a 1.25D array, a 1.75D array, an annular array, a multidimensional array, a wobbler array, combinations thereof, or any other now known or later developed array. The transducer elements transduce between acoustic and electric energies. The transducer 806 connects with the transmit beamformer 802 and the receive beamformer 804 through a transmit/receive switch, but separate or other connections may be used in other cases.

The transmit and receive beamformers 802, 804 are configured for scanning with the transducer 14. The transmit beamformer 802, using the transducer 806, transmits one or more beams to scan a region. Various scan formats may be used. The receive beamformer 804 samples the receive beams at different depths.

In some cases, the transmit beamformer 802 is or includes a processor, delay, filter, waveform generator, memory, phase rotator, digital-to-analog converter, amplifier, combinations thereof or any other now known or later developed transmit beamformer components. Using filtering, delays, phase rotation, digital-to-analog conversion and amplification, the desired transmit waveform is generated. Other waveform generators may be used, such as switching pulsers or waveform memories.

The transmit beamformer 802 may be configured as a plurality of channels for generating electrical signals of a transmit waveform for each element of a transmit aperture on the transducer 806. The waveforms may be unipolar, bipolar, stepped, sinusoidal or other waveforms of a desired center frequency or frequency band with one, multiple or fractional number of cycles. The waveforms may have relative delay and/or phasing and amplitude for focusing the acoustic energy. The transmit beamformer 802 may include a controller for altering an aperture (e.g. the number of active elements), an apodization profile (e.g., type or center of mass) across the plurality of channels, a delay profile across the plurality of channels, a phase profile across the plurality of channels, center frequency, frequency band, waveform shape, number of cycles and combinations thereof. A transmit beam focus is generated based on these beamforming parameters.

The receive beamformer 804 is or includes a preamplifier, filter, phase rotator, delay, summer, base band filter, processor, buffers, memory, combinations thereof or other now known or later developed receive beamformer components. The receive beamformer 804 is configured into a plurality of channels for receiving electrical signals representing echoes or acoustic energy impinging on the transducer 806. A channel from each of the elements of the receive aperture within the transducer 804 connects to an amplifier and/or delay. An analog-to-digital converter digitizes the amplified echo signal. The digital radio frequency received data is demodulated to a base band frequency. Any receive delays, such as dynamic receive delays, and/or phase rotations are then applied by the amplifier and/or delay. A digital or analog summer combines data from different channels of the receive aperture to form one or a plurality of receive beams. The summer is a single summer or cascaded summer. In one embodiment, the beamform summer is operable to sum in-phase and quadrature channel data in a complex manner such that phase information is maintained for the formed beam. Alternatively, the beamform summer sums data amplitudes or intensities without maintaining the phase information.

The receive beamformer 804 is operable to form receive beams in response to the transmit beams. For example, the receive beamformer 804 receives one, two, or more (e.g., 30, 40, or 50) receive beams in response to each transmit beam. The receive beams are collinear, parallel and offset or nonparallel with the corresponding transmit beams. The receive beamformer 804 outputs spatial samples representing different spatial locations of a scanned region. Once the channel data is beamformed or otherwise combined to represent spatial locations along the scan lines, the data is converted from the channel domain to the image data domain. The phase rotators, delays, and/or summers may be repeated for parallel receive beamformation. One or more of the parallel receive beamformers may share parts of channels, such as sharing initial amplification.

In the example of FIG. 8, the system 800 includes a computing system 808 having a processor 810, a memory 812, and a display 814. The computing system 808 may be integrated with the ultrasound imaging system to any desired extent. The processor 810 is in communication with the memory 812 for execution of instructions stored in the memory 812. In this example, scan data input instructions, scan data analysis instructions, segmentation instructions, and boundary identification instructions are stored in the memory 812. Additional, fewer, or alternative instructions are provided. For instance, the instructions may be integrated with one another to any desired extent.

The execution of the instructions stored in the memory 812 may cause the processor 810 to implement one or more acts of the above-described methods. For instance, upon execution of the scan data input instructions, the processor 810 is configured to obtain scan data representative of a two-dimensional slice through the optic nerve sheath and a globe from which the optic nerve extends. Upon execution of the scan data analysis instructions, the processor 810 is configured to analyze the scan data to find an anterior-posterior position of a globe-optic nerve interface point. Upon execution of the segmentation instructions, the processor 810 is configured to implement a segmentation procedure to generate a super-pixel representation of the scan data. Upon execution of the boundary identification instructions, the processor 810 is configured to process the super-pixel representation of the scan data at an offset from the anterior-posterior position of the globe-optic nerve interface point to determine boundary positions of the optic nerve sheath, and calculate the diameter of the optic nerve sheath based on the determined boundary positions. The configuration of the processor 810 via these instructions may vary as described above. For instance, the segmentation procedure implemented by the processor 810 may include a k-means clustering procedure and/or another segmentation procedure. The execution of the segmentation instructions may further configure the processor 810 to discard super-pixels below a threshold size. The execution of the boundary identification instructions further configures the processor 810 to (i) determine a lateral position of the globe-optic nerve interface point at the offset based on a minimum between peaks in the super-pixel representation of the scan data, (ii) compute a derivative of the super-pixel representation of the scan data at the offset, and (iii) find a pair of peaks in the derivative of the super-pixel representation of the scan data, each peak of the pair of peaks being disposed on a respective side of the lateral position.

The processor 810 may include one or more processors or processing units. In some cases, the processor 810 is or includes a digital signal processor, a general processor, an application specific integrated circuit, a field programmable gate array, a control processor, digital circuitry, analog circuitry, a graphics processing unit, combinations thereof or other now known or later developed device for implementing calculations, algorithms, programming or other functions. The processor 810 may or may not be configured to execute instructions provided in the memory 812, or a different memory, for directed to controlling the imaging system and/or rendering of the captured ultrasound scan data.

The memory 812 may include one or more memories. In some cases, the memory 812 is or includes video random access memory, random access memory, removable media (e.g. diskette or compact disc), a hard drive, a database, or other memory device for storing instructions, scan data, and/or other data. The memory 812 may be operable to store signals responsive to multiple transmissions along a substantially same scan line. The memory 812 is operable to store ultrasound data in various formats.

The display 814 is or includes a CRT, LCD, plasma, projector, monitor, printer, touch screen, or other now known or later developed display device. The display 814 receives RGB or other color data and outputs an image. The image may be a gray scale or color image. The image represents the region of the patient scanned by the transducer 806 and other components of the imaging system.

The instructions for implementing the processes, methods and/or techniques discussed above are provided on computer-readable storage media or memories, such as a cache, buffer, RAM, removable media, hard drive or other computer readable storage media. In one embodiment, the instructions are for volumetric quantification. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

Experimental Results. An example of the disclosed method was applied to 50 de-identified videos of 25 traumatic injured patients. Ultrasound images of both eyes were captured for each patient. The results of the disclosed method were compared with ground truth measurements, which were measurements from two experts. The correlation between two experts' measurements was also calculated. It should be noted that the individuals performing the manual measurements were blinded to each other's measurements as well as the algorithm measurement. Four types of comparisons were implemented. In the first one, the average error between the proposed method and the ground truth was calculated using the equation below.

$$e = \frac{1}{{n}_{u}} {\sum }_{k = 1}^{{n}_{u}} \vert 1 - \frac{{ONSD}_{1} ( k )}{{ONSD}_{2} ( k )} \vert \times 100$$

In Equation (5), nᵤ is the number of ultrasound images. Also, ONSD₁ and ONSD₂ are the ONSD measurements from two sources. For instance, for comparing the results of the proposed method with the ground truth, ONSD₁ and ONSD₂ are the algorithm results and the average of two experts' measurements respectively. Moreover, for comparing two experts' measurement, ONSD₁ and ONSD₂ are the measurements from each expert. The average percentage of error between the results of the algorithm and the average manual measurements was 5.52%. This error was 4.74% between two experts' measurements. The difference between these two errors show that the disclosed methods and systems can calculate the ONSD accurately. In the second comparison, the mean square error (MSE) was calculated using the equation below, where ∥.∥₂ is the norm-2.

$$M S E = \frac{1}{{n}_{u}} {‖ {ONSD}_{1} - {ONSD}_{2} ‖}_{2}$$

The MSE between the algorithm results and the average of two experts' measurements was 0.0018, while the MSE between two experts' measurement was 0.0016. In the third comparison, intraclass correlation coefficient (ICC) was calculated, which shows the similarity between two quantitative measurements. The ICC between the algorithm results and the average of two experts' measurements was 0.70, while the ICC between two experts' measurement was 0.80. In the last comparison, the student t-test was performed, which is a statistical test to test the null hypothesis that the means of two measurements are not different. Using the confidence interval of 95%, the p-value of the t-test between the algorithm results and the average of two experts' measurements was 0.45, while this value for the t-test between the two experts' measurements was 0.26. These p-values show that the t-test doesn't reject the null hypothesis. All of the four aforementioned comparisons indicate strong correlation between the proposed method' s results and the ground truth.

The methods and systems described above may be used to calculate additional or alternative parameters or characteristics regarding the optic nerve sheath. For instance, the area inside the optic nerve sheath for each frame (i.e., two-dimensional slice) may be calculated. The area may be bounded laterally by the optic nerve sheath, and from the globe-optic nerve interface point to the 3 mm depth (or another depth) in the anterior-posterior dimension. The area may accordingly have a semi-circular shape, as shown by the lines superimposed on the image of FIG. 2. The area may then be used to estimate the ICP level. The correlation between the area and the ICP level may be stored in a look-up table or other database, as described above.

Described above are methods and systems for automatically and non-invasively measuring optic nerve sheath diameter from scan data, such as ultrasound imaging data. As a non-invasive procedure, the measurement techniques of the disclosed methods and systems reduce the costs associated with efforts to use sheath diameter as a predictor of ICP increase. The automated nature of the measurement techniques of the disclosed methods and systems avoid the time consuming and error prone aspects of manual measurement techniques. In some cases, the disclosed methods and systems implement image processing in which the optic nerve sheath diameter is measured automatically by removing noise from the image scans, detecting a region of interest using a line integral method, and analyzing super-pixels generated via image segmentation. Results of tests of the disclosed method did not differ substantially from manual measurements conducted by two experts. The average percentage of error between the disclosed method and the experts' measurements did not substantially differ from the error between the respective measurements of the two experts.

Even though intracranial-pressure monitoring is a standard care for severe traumatic injured patients, using such invasive devices might be associated with worsening of survival and there might be complications following the placement of ICP sensors. Therefore, the non-invasive monitoring provided by the disclosed methods and systems can prevent secondary complications. It has been shown that there is a correlation between the ICP elevation and the optic nerve sheath diameter. The disclosed methods and systems calculate this diameter using image processing techniques. In one example, images are first denoised, and a region of interest is identified using a line-integral method. A super-pixel segmentation method is then applied to the subset of scan data in the region of interest. After that, the row or line of segmented scan data at 3 mm below the globe is used to measure the diameter of the nerve sheath. The diameter may be measured by computing the derivative of that row and finding the peaks in the derivative.

The present disclosure has been described with reference to specific examples that are intended to be illustrative only and not to be limiting of the disclosure. Changes, additions and/or deletions may be made to the examples without departing from the spirit and scope of the disclosure.

The foregoing description is given for clearness of understanding only, and no unnecessary limitations should be understood therefrom.

What is claimed is:

1. A method of determining a diameter of a sheath of an optic nerve, the method comprising:
obtaining, by a processor, scan data representative of the optic nerve sheath;
analyzing, by the processor, the scan data to find a position of a globe-optic nerve interface point;
segmenting, by the processor, the scan data;
processing, by the processor, the segmented scan data at an offset from the position of the globe-optic nerve interface point to determine boundary positions of the optic nerve sheath; and
calculating, by the processor, the diameter of the optic nerve sheath based on the determined boundary positions.

2. The method of claim 1, wherein processing the segmented scan data comprises:
finding peaks in the segmented scan data at the offset; and
determining a location of a minimum between the found peaks.

3. The method of claim 1, wherein processing the segmented scan data comprises processing the segmented scan data comprises computing a derivative of the segmented scan data at the offset.

4. The method of claim 1, wherein processing the segmented scan data comprises:
determining a lateral position of the globe-optic nerve interface point at the offset based on a minimum between peaks in the segmented scan data;
computing a derivative of the segmented scan data at the offset; and
finding a pair of peaks in the derivative of the segmented scan data, each peak of the pair of peaks being disposed on a respective side of the lateral position.

5. The method of claim 4, wherein finding the first and second peaks comprises disregarding peaks in the derivative greater than a threshold.

6. The method of claim 4, wherein finding the first and second peaks further comprises, after disregarding the peaks greater than the threshold:
finding a negative peak closest to the lateral position of the globe-optic nerve interface point; and
finding a positive peak closest to the lateral position of the globe-optic nerve interface point.

7. The method of claim 1, wherein analyzing the scan data comprises:
computing a line integral of the scan data at each anterior-posterior position of the scan data; and
finding a maximum of the line integral to determine the position of the globe-optic nerve interface point.

8. The method of claim 7, wherein the line integral is a first line integral, the method further comprising:
computing a second line integral of the scan data at each lateral position of the scan data; and
determining a subset of the scan data corresponding with a region of interest based on the first line integral and the second line integral;
wherein segmenting the scan data is implemented on the determined subset of the scan data.

9. The method of claim 8, wherein:
the scan data comprises a plurality of frames; and
the method further comprises disregarding one or more frames of the plurality of frames based on whether the first and second line integrals present peaks indicative of the globe and the optic nerve such that analyzing the scan data, segmenting the scan data, processing the segmented scan data, and calculating the diameter are repeated for the scan data of each remaining frame of the plurality of frames.

10. The method of claim 1, wherein the scan data comprises two-dimensional slice data, the two-dimensional slice data being representative of a slice through the globe and the optic nerve.

11. The method of claim 1, wherein processing the segmented scan data comprises selecting a line of the scan data located about 3 millimeters in a posterior direction from the globe-optic nerve interface point as a subset of the segmented scan data at the offset to be processed.

12. The method of claim 1, wherein obtaining the scan data comprises:
capturing ultrasound scan data;
cropping the ultrasound data; and
removing noise from the cropped ultrasound scan data to generate the scan data;
wherein removing the noise comprises implementing a filtering procedure configured to preserve edges in the cropped ultrasound scan data.

13. The method of claim 1, wherein:
the scan data comprises a plurality of frames;
analyzing the scan data, segmenting the scan data, processing the segmented scan data, and calculating the diameter are repeated for the scan data of each frame of the plurality of frames; and
the method further comprises compiling the calculated diameters of the optic nerve sheath for the plurality of frames to determine a value for the diameter of the optic nerve sheath.

14. A method of determining an assessment of intracranial pressure comprising the method of claim 13, and further comprising determining an intracranial pressure level based on the value for the diameter and based on a database correlating diameter values with corresponding levels of intracranial pressure.

15. A system of determining a diameter of an optic nerve sheath, the system comprising:
a memory in which scan data input instructions, scan data analysis instructions, segmentation instructions, and boundary identification instructions are stored; and
a processor in communication with the memory and configured to
upon execution of the scan data input instructions, obtain scan data representative of a two-dimensional slice through the optic nerve sheath and a globe from which the optic nerve extends;
upon execution of the scan data analysis instructions, analyze the scan data to find an anterior-posterior position of a globe-optic nerve interface point;
upon execution of the segmentation instructions, implement a segmentation procedure to generate a super-pixel representation of the scan data; and
upon execution of the boundary identification instructions, process the super-pixel representation of the scan data at an offset from the anterior-posterior position of the globe-optic nerve interface point to determine boundary positions of the optic nerve sheath, and calculate the diameter of the optic nerve sheath based on the determined boundary positions.

16. The system of claim 15, wherein the segmentation procedure comprises a k-means clustering procedure.

17. The system of claim 15, wherein the execution of the segmentation instructions further configures the processor to discard super-pixels below a threshold size.

18. The system of claim 15, wherein the execution of the boundary identification instructions further configures the processor to
determine a lateral position of the globe-optic nerve interface point at the offset based on a minimum between peaks in the super-pixel representation of the scan data;
compute a derivative of the super-pixel representation of the scan data at the offset; and
find a pair of peaks in the derivative of the super-pixel representation of the scan data, each peak of the pair of peaks being disposed on a respective side of the lateral position.

19. A computer readable storage medium having stored therein data representing instructions executable by a programmed processor for determining a diameter of an optic nerve sheath, the storage medium comprising instructions for:
obtaining scan data representative of a two-dimensional slice through the optic nerve sheath and a globe from which the optic nerve extends;
analyzing the scan data to find a position of a globe-optic nerve interface point;
implementing a segmentation procedure, the segmentation procedure being configured to generate a super-pixel representation of the scan data;
processing the super-pixel representation of the scan data at an offset from the anterior-posterior position of the globe-optic nerve interface point to determine boundary positions of the optic nerve sheath; and
calculating the diameter of the optic nerve sheath based on the determined boundary positions.

20. The computer readable storage medium of claim 19, wherein processing the super-pixel representation of the scan data comprises:
determining a lateral position of the globe-optic nerve interface point at the offset based on a minimum between peaks in the super-pixel representation of the scan data;
computing a derivative of the super-pixel representation of the scan data at the offset; and
finding a pair of peaks in the derivative of the super-pixel representation of the scan data, each peak of the pair of peaks being disposed on a respective side of the lateral position.
